(19) United States
(12) Patent Application Publication
(10) Pub. No.: US 2012/0118198 A1
(43) Pub. Date: May 17, 2012

(54) ELECTRONIC INKS

(76) Inventors: Zhang-Lin Zhou, Palo Alto, CA (US); Jong-Souk Yeo, Corvallis, OR (US); Jeffrey Todd Mabeck, Corvallis, OR (US); Richard H. Henze, San Carlos, CA (US)

(21) Appl. No.: 13/386,896

(22) PCT Filed: Oct. 16, 2009
(86) PCT No.: PCT/US2009/060975

§ 371 Date: Jan. 24, 2012

(51) Int. Cl. C09D 11/00; C07C 233/34; C07C 69/34; C07C 43/04; C07C 211/13

(52) U.S. Cl. 106/31.6; 106/31.13; 568/672; 564/512; 560/190; 564/224

(57) ABSTRACT

Electronic inks are disclosed herein. One embodiment of the electronic ink includes a non-polar carrier fluid, a plurality of negatively charged colorant particles dispersed in the non-polar carrier fluid, a small molecular additive, and a charge director. Each of the negatively charged colorant particles includes a surface modified with an acidic functional group. The small molecular additive has at least two branches, each of which includes nitrogen or oxygen atoms.

### BACKGROUND

The present disclosure, relates generally to electronic inks.

Electronic inks are commonly used in electronic displays. Such electronic inks often include charged colorant particles that, in response to an applied electric field, rearrange within a viewing area of the display to produce desired images.

### BRIEF DESCRIPTION OF THE DRAWINGS

Features and advantages of embodiments of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 depicts an example of a mechanism for forming negatively charged colorant particles for an embodiment of an electronic ink;

FIGS. 2A and 2B depict examples of negatively charged colorant particles for an embodiment of an electronic ink;

FIG. 3 depicts another example of a mechanism for forming negatively charged colorant particles for an embodiment of an electronic ink;

FIG. 4 depicts yet another example of a mechanism for forming negatively charged colorant particles for an embodiment of an electronic ink; and

FIG. 5 depicts still one more example of a mechanism for forming negatively charged colorant particles for an embodiment of an electronic ink.

### DETAILED DESCRIPTION

Embodiment(s) of the electronic ink as disclosed herein may be used in various electronic displays, such as, for example, in electro-optical displays. Such electro-optical displays include those that are driven by electrophoresis, electro-convective flow, and/or other electrokinetic effects or combinations of electrokinetic effects. Such inks can be used in displays with in-plane shutter architectures, where colorant particles are moved laterally into and out of a field of view in a pixel or sub-pixel cell in a display. Electro-optical display architectures that include at least one layer of the electronic ink of the instant disclosure are capable of enabling addressing of every available color at every location in the display. This tends to produce brighter and more colorful images.

The foregoing may be accomplished by using an electronic ink in the display, where the electronic ink includes negatively charged colorant particles dispersed in a non-polar carrier fluid. The colorant particles have one or more molecular additives adsorbed onto surfaces thereof. Without being bound to any theory, it is believed that the molecular additives sterically hinder the colorant particle. In other words, when the molecular additive(s) is/are attached to the colorant particle, such particle is rendered sterically hindered. It is further believed that the adsorption of the molecular additives is accomplished through hydrogen bonding between acid modified surfaces of the particles and oxygen and/or nitrogen present in the additive(s). Such hydrogen bonding is believed to contribute, at least in part, to an increase in the hydrophobicity of the colorant particle surfaces, thereby improving the dispersibility of the colorant particle in the non-polar fluid. Such hydrogen bonding is further believed to improve the chargeability of the colorant particles, which tends to improve the switching speed of the display, as well as clearance and/or compaction of the colorant particles onto electrodes included in the display architecture.

In other embodiments disclosed herein, the colorant particles include negatively charged carbon black pigment particles coated with an inorganic insulating layer such as, e.g., silica. Such negatively charged carbon black pigment particles are generally stable, charged, and may be used in electronic devices without creating electrical shorting. The performance of the negatively charged carbon black pigment particles may be further improved by adsorbing a molecular additive on the surfaces of the particles. Again, such molecular additives increase the hydrophobicity of the particle surfaces (through hydrogen bonding of the additive to the silica coated pigment surface), thereby improving the dispersibility of the carbon black pigment in its carrier fluid, as well as its chargeability. As mentioned hereinabove, the addition of the molecular additive(s) to the colorant particle surface also renders the carbon black pigment particles sterically hindered.

The embodiment(s) of the electronic ink generally include a non-polar carrier fluid (i.e., a fluid having a low dielectric constant k such as, e.g., less than about 20, or, in some cases, less than about 2). Such fluids tend to reduce leakages of electric current when driving the display, as well as increase the electric field present in the fluid. As used herein, the “carrier fluid” is a fluid or medium that fills up a viewing area defined in an electro-optical display and is generally configured as a vehicle to carry colorant particles therein. In response to a sufficient electric potential or field applied to the colorant particles while driving electrodes of the display, the colorant particles tend to move and/or rotate to various spots within the viewing area in order to produce a desired visible effect in the display cell to display an image. The non-polar carrier fluid includes, for example, one or more non-polar solvents selected from hydrocarbons, halogenated or partially halogenated hydrocarbons, oxygenated fluids, siloxanes, and/or silicones. Some specific examples of non-polar solvents include perchloroethylene, halocarbons, cyclohexane, dodecane, mineral oil, isoparaffinic fluids, cyclopentasiloxane, cyclohexasiloxane, and combinations thereof.

The colorant particles are dispersed in the carrier fluid. As used herein, the term “colorant particles” refers to particles that produce a color. Some non-limiting examples of suitable colorant particles include pigment particles, a combination of pigment particles and a dye, nanoparticle pigment dispersions, polymer particles colored with dye molecules, or the like. In a non-limiting example, the colorant particles are selected from pigment particles that are self-dispersible in the non-polar carrier fluid. It is to be understood, however, that non-dispersible pigment particles may otherwise be used so long as the electronic ink includes one or more suitable dispersants. Such dispersants include hyperdispersants such as those of the SOLSPERSE® series manufactured by Lubrizol Corp., Wickliffe, Ohio (e.g., SOLSPERSE® 3000, SOLSPERSE® 8000, SOLSPERSE® 9000, SOLSPERSE® 11200, SOLSPERSE®13840, SOLSPERSE®16000, SOLSPERSE®17000, SOLSPERSE® 18000, SOLSPERSE® 19000, SOLSPERSE® 21000, and SOLSPERSE® 27000); various dispersants manufactured by BYK-chemie, Gmbh, Germany, (e.g., DISPERBYK® 110, DISPERBYK® 163, DISPERBYK® 170, and DISPERBYK® 180); various dispersants manufactured by Evonik Goldschmidt GMBH LLC, Germany, (e.g., TEGO® 630, TEGO® 650, TEGO® 651, TEGO® 655, TEGO® 685, and TEGO® 1000); and various dispersants manufactured by Sigma-Aldrich, St. Louis, Mo., (e.g., SPAN® 20, SPAN® 60, SPAN® 80, and SPAN® 85).

The pigment particles are selected from organic or inorganic pigments, and have an average particle size ranging from about 1 nm to about 10 μm. In some instances, the average particle size ranges from about 50 nm to about 1 μm. Such organic or inorganic pigment particles may be selected from black pigment particles, yellow pigment particles, magenta pigment particles, red pigment particles, cyan pigment particles, blue pigment particles, green pigment particles, orange pigment particles, brown pigment particles, and white pigment particles. In some instances, the organic or inorganic pigment particles may include spot-color pigment particles, which are formed from a combination of a predefined ratio of two or more primary color pigment particles.

A non-limiting example of a suitable inorganic black pigment includes carbon black. Examples of carbon black pigments include those manufactured by Mitsubishi Chemical Corporation, Japan (such as, e.g., carbon black No. 2300, No. 900, MCF88, No. 33, No. 40, No. 45, No. 52, MA7, MA8, MA100, and No. 2200B); various carbon black pigments of the RAVEN® series manufactured by Columbian Chemicals Company, Marietta, Ga., (such as, e.g., RAVEN® 5750, RAVEN® 5250, RAVEN® 5000, RAVEN® 3500, RAVEN® 1255, and RAVEN® 700); various carbon black pigments of the REGAL® series, the MOGUL® series, or the MONARCH® series manufactured by Cabot Corporation, Boston, Mass., (such as, e.g., REGAL® 400R, REGAL® 330R, REGAL® 660R, MOGUL® L, MONARCH® 700, MONARCH® 800, MONARCH® 880, MONARCH® 900, MONARCH® 1000, MONARCH® 1100, MONARCH® 1300, and MONARCH® 1400); and various black pigments manufactured by Evonik Degussa Corporation, Parsippany, N.J., (such as, e.g., Color Black FW1, Color Black FW2, Color Black FW2V, Color Black FW18, Color Black FW200, Color Black S150, Color Black S160, Color Black S170, PRINTEX® 35, PRINTER® U, PRINTEX® V, PRINTEX® 140U, Special Black 5, Special Black 4A, and Special Black 4). A non-limiting example of an organic black pigment includes aniline black, such as C.I. Pigment Black 1.

Some non-limiting examples of suitable yellow pigments include C.I. Pigment Yellow 1, C.I. Pigment Yellow 2, C.I. Pigment Yellow 3, C.I. Pigment Yellow 4, C.I. Pigment Yellow 5, C.I. Pigment Yellow 6, C.I. Pigment Yellow 7, C.I. Pigment Yellow 10, C.I. Pigment Yellow 11, C.I. Pigment Yellow 12, C.I. Pigment Yellow 13, C.I. Pigment Yellow 14, C.I. Pigment Yellow 16, C.I. Pigment Yellow 17, C.I. Pigment Yellow 24, C.I. Pigment Yellow 34, C.I. Pigment Yellow 35, C.I. Pigment Yellow 37, C.I. Pigment Yellow 53, C.I. Pigment Yellow 55, C.I. Pigment Yellow 65, C.I. Pigment Yellow 73, C.I. Pigment Yellow 74, C.I. Pigment Yellow 75, C.I. Pigment Yellow 81, C.I. Pigment Yellow 83, C.I. Pigment Yellow 93, C.I. Pigment Yellow 94, C.I. Pigment Yellow 95, C.I. Pigment Yellow 97, C.I. Pigment Yellow 98, C.I. Pigment Yellow 99, C.I. Pigment Yellow 108, C.I. Pigment Yellow 109, C.I. Pigment Yellow 110, C.I. Pigment Yellow 113, C.I. Pigment Yellow 114, C.I. Pigment Yellow 117, C.I. Pigment Yellow 120, C.I. Pigment Yellow 124, C.I. Pigment Yellow 128, C.I. Pigment Yellow 129, C.I. Pigment Yellow 133, C.I. Pigment Yellow 138, C.I. Pigment Yellow 139, C.I. Pigment Yellow 147, C.I. Pigment Yellow 151, C.I. Pigment Yellow 153, C.I. Pigment Yellow 154, C.I. Pigment Yellow 167, C.I. Pigment Yellow 172, and C.I. Pigment Yellow 180.

Non-limiting examples of suitable magenta or red organic pigments include C.I. Pigment Red 1, C.I. Pigment Red 2, C.I. Pigment Red 3, C.I. Pigment Red 4, C.I. Pigment Red 5, C.I. Pigment Red 6, C.I. Pigment Red 7, C.I. Pigment Red 8, C.I. Pigment Red 9, C.I. Pigment Red 10, C.I. Pigment Red 11, C.I. Pigment Red 12, C.I. Pigment Red 14, C.I. Pigment Red 15, C.I. Pigment Red 16, C.I. Pigment Red 17, al. Pigment Red 18, C.I. Pigment Red 19, C.I. Pigment Red 21, C.I. Pigment Red 22, C.I. Pigment Red 23, C.I. Pigment Red 30, C.I. Pigment Red 31, C.I. Pigment Red 32, C.I. Pigment Red 37, C.I. Pigment Red 38, C.I. Pigment Red 40, C.I. Pigment Red 41, C.I. Pigment Red 42, C.I. Pigment Red 48(Ca), C.I. Pigment Red 48(Mn), C.I. Pigment Red 57(Ca), C.I. Pigment Red 57:1, C.I. Pigment Red 88, C.I. Pigment Red 112, C.I. Pigment Red 114, C.I. Pigment Red 122, C.I. Pigment Red 123, C.I. Pigment Red 144, C.I. Pigment Red 146, C.I. Pigment Red 149, C.I. Pigment Red 150, C.I. Pigment Red 166, C.I. Pigment Red 168, C.I. Pigment Red 170, C.I. Pigment Red 171, C.I. Pigment Red 175, C.I. Pigment Red 176, C.I. Pigment Red 177, C.I. Pigment Red 178, C.I. Pigment Red 179, C.I. Pigment Red 184, C.I. Pigment Red 185, C.I. Pigment Red 187, C.I. Pigment Red 202, C.I. Pigment Red 209, C.I. Pigment Red 219, C.I. Pigment Red 224, C.I. Pigment Red 245, C.I. Pigment Violet 19, C.I. Pigment Violet 23, C.I. Pigment Violet 32, C.I. Pigment Violet 33, C.I. Pigment Violet 36, C.I. Pigment Violet 38, C.I. Pigment Violet 43, and C.I. Pigment Violet 50.

Non-limiting examples of blue or cyan organic pigments include C.I. Pigment Blue 1, C.I. Pigment Blue 2, C.I. Pigment Blue 3, C.I. Pigment Blue 15, C.I. Pigment Blue 15:3, C.I. Pigment Blue 15:34, C.I. Pigment Blue 15:4, C.I. Pigment Blue 16, C.I. Pigment Blue 18, C.I. Pigment Blue 22, C.I. Pigment Blue 25, C.I. Pigment Blue 60, C.I. Pigment Blue 65, C.I. Pigment Blue 66, C.I. Vat Blue 4, and C.I. Vat Blue 60.

Non-limiting examples of green organic pigments include C.I. Pigment Green 1, C.I. Pigment Green2, C.I. Pigment Green, 4, C.I. Pigment Green 7, C.I. Pigment Green 8, C.I. Pigment Green 10, C.I. Pigment Green 36, and C.I. Pigment Green 45.

Non-limiting examples of brown organic pigments include C.I. Pigment Brown 1, C.I. Pigment Brown 5, C.I. Pigment Brown 22, C.I. Pigment Brown 23, C.I. Pigment Brown 25, and C.I. Pigment Brown, C.I. Pigment Brown 41, and C.I. Pigment Brown 42.

Non-limiting examples of orange organic pigments include C.I. Pigment Orange 1, C.I. Pigment Orange 2, C.I. Pigment Orange 5, C.I. Pigment Orange 7, C.I. Pigment Orange 13, C.I. Pigment Orange 15, C.I. Pigment Orange 16, C.I. Pigment Orange 17, C.I. Pigment Orange 19, C.I. Pigment Orange 24, C.I. Pigment Orange 34, C.I. Pigment Orange 36, C.I. Pigment Orange 38, C.I. Pigment Orange 40, C.I. Pigment Orange 43, and C.I. Pigment Orange 66.

In an embodiment, each of the colorant particles has adsorbed thereon a molecular additive. Without being bound to any theory, it is believed that the molecular additive is adsorbed on the surface and bonds to the surface via hydrogen bonding. More specifically, it is believed that a hydrogen bond forms between the oxygen or nitrogen atoms present in the molecular chain of the molecular additive and hydroxyl functional groups of the colorant particle. The oxygen or nitrogen atoms act as a hydrogen bond acceptor for the hydroxyl group hydrogen bond donor. The hydrogen bonding is believed to increase the hydrophobicity of the particle surface at least in part because the hydroxide functional groups, which are hydrophilic, introduce hydrophobic acrylates to the particle when they adsorb the molecular additive. It is believed that such increased hydrophobicity of the colorant particle improves the dispersibility of the colorant in the non-polar carrier fluid, as well as the chargeability and the stability of the colorant particle. Such improved chargeability and stability tends to improve the switching speed and clearance and/or compaction of the colorant particle on electrode surfaces of the display.

The molecular additive adsorbed on the surface of the colorant particle is small and generally has a branched molecular structure. A “small” molecular additive is one having a molecular weight that is less than 2000. In an embodiment, the small molecular additive includes at least two branches, each including nitrogen or oxygen atoms in the chain of the respective branch. In other embodiments, the small molecular additive includes two branches, three branches, or four branches. Some non-limiting examples of suitable small molecular additives have the following base structures:

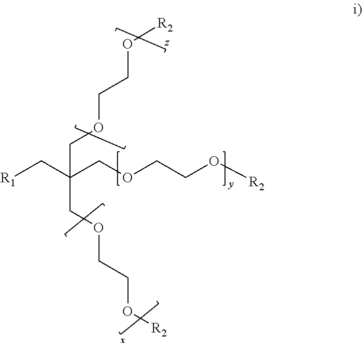

where R₁ and R₂ are each independently selected from an alkyl group, a branched alkyl group, an aliphatic group, an aromatic acyl group, an alkenyl group, and a branched alkenyl group, and x, y, and z are each selected from any whole number ranging from 0 to 10;

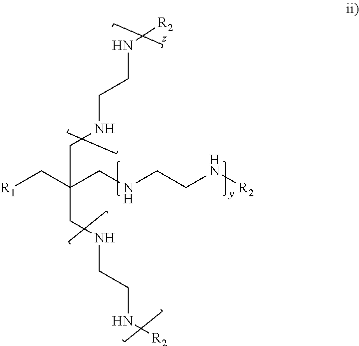

where R₁ and R₂ are each independently selected from an alkyl group, a branched alkyl group, an aliphatic group, an aromatic acyl group, an alkenyl group, and a branched alkenyl group, and x, y, and z are each selected from any whole number ranging from 0 to 10;

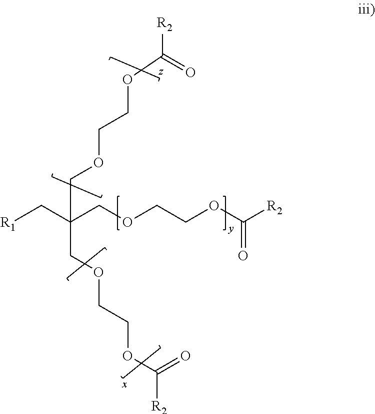

where R₁ and R₂ are each independently selected from an alkyl group, a branched alkyl group, an alkenyl group, or a branched alkenyl group, and x, y, and z are each selected from a whole number ranging from 0 to 10;

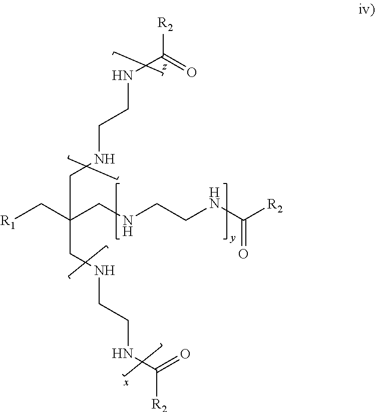

where R₁ and R₂ are each independently selected from an alkyl group, a branched alkyl group, an alkenyl group, or a branched alkenyl group, and x, y, and z are each selected from a whole number ranging from 0 to 10;

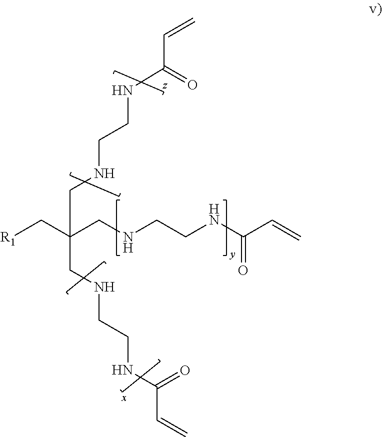

where R₁ is selected from an alkyl group, a branched alkyl group, an alkenyl group, or a branched alkenyl group, and x, y, and z are selected from a whole number ranging from 0 to 10; and

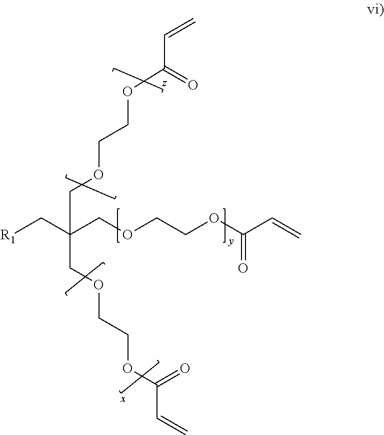

where R₁ is selected from an alkyl group, a branched alkyl group, an alkenyl group, or a branched alkenyl group, and x, y, and z are selected from a whole number ranging from 0 to 10.

Some specific examples of suitable small molecular additives that may be used in the electronic ink of some of the embodiments disclosed herein are as follows:

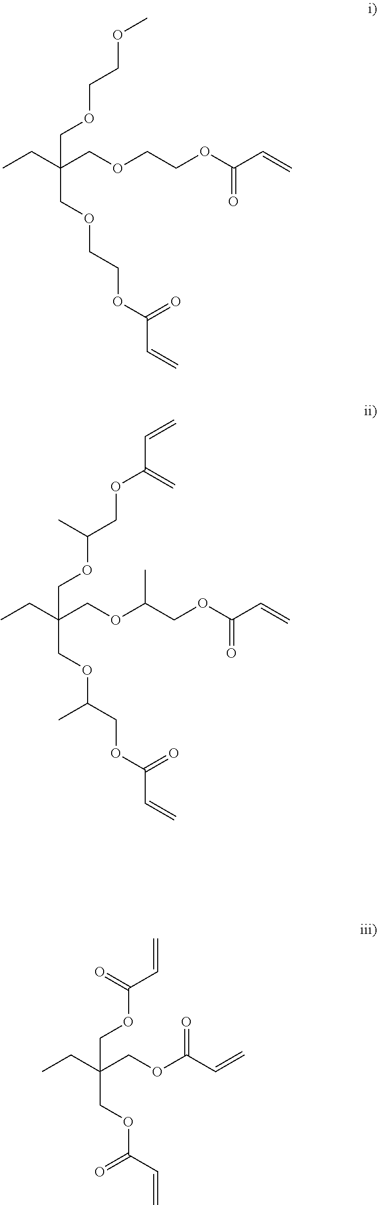

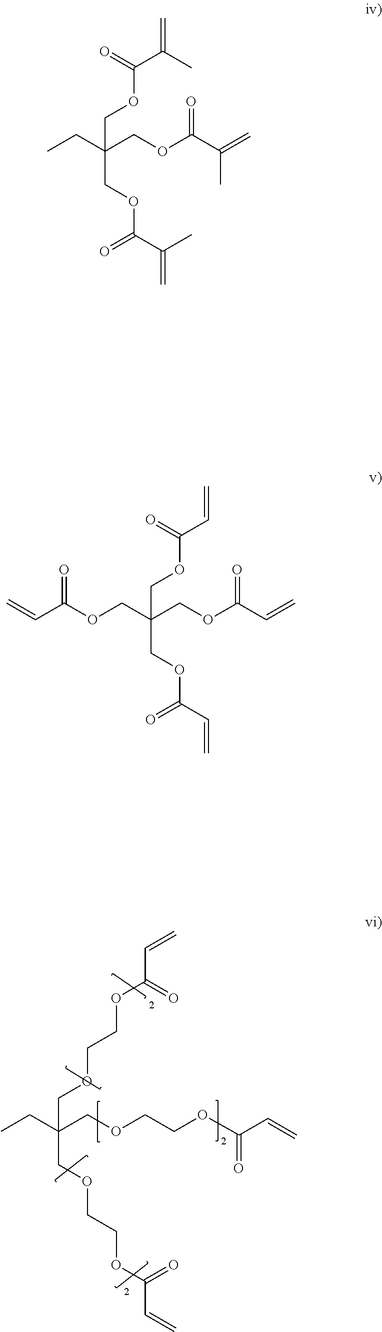

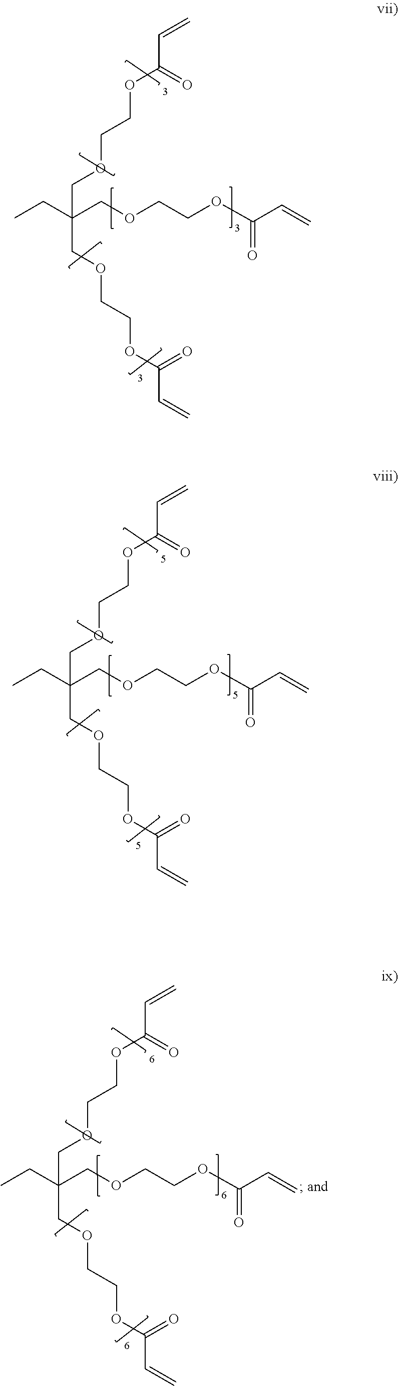

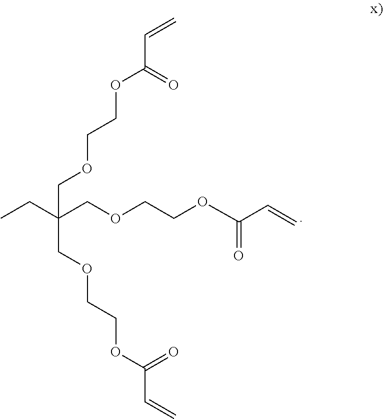

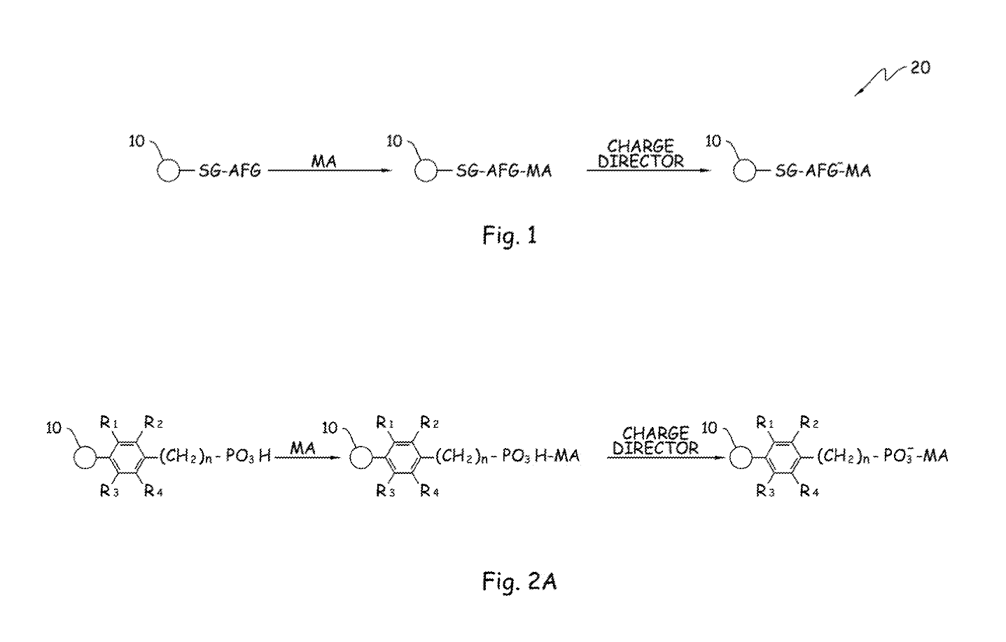

As mentioned herein, these small molecules are adsorbed to the surface of negatively charged colorant particles. An example of a mechanism for forming the negatively charged colorant particles 20 is shown in FIG. 1. This mechanism includes modifying the surface of the colorant particle (identified by a sphere labeled with reference numeral 10 in FIG. 1) with one or more acidic functional groups (AFG). Non-limiting examples of suitable acidic functional groups include OH, SH, COOH, CSSH, COSH, SO₃H, PO₃H, OSO₃H, OPO₃H, or combinations thereof.

The modification of the colorant particle 10 surface may be accomplished by connecting the acidic functional group (AFG) to the particle 10 surface via a spacing group (SG). The spacing group (SG) is used when the acidic functional group (AFG) cannot be introduced directly onto the particle 10 surface (e.g., when the AFG would render the particle 10 instable). The spacing group (SG) may be selected from any substituted or unsubstituted aromatic molecular structure such as benzenes, substituted benzenes, naphthalenes, molecules including aliphatic chains, substituted naphthalenes, and/or hetero-aromatic structures (such as, e.g., pyridines, pyrimidines, triazines, furans, and the like). The spacing group (SG) may otherwise be an inorganic coating established on the colorant 10 surface such as, e.g., SiO₂ coatings, TiO₂ coatings, HfO₂ coatings, Al₂O₃ coatings, ZrO₂ coatings, ZnO coatings, Si₃N₄ coatings, and/or the like. In a non-limiting example, a single acidic functional group (AFG) is connected to the spacing group (SG) (as shown in the mechanism depicted in FIG. 1). In other non-limiting examples, two or more of the acidic functional groups (AFG) may be connected to a single spacing group (SG) (not shown in the figures).

Once the surface of the colorant particle 10 has been modified with the acidic functional group(s) (AFG), the mechanism further includes adsorbing the molecular additive (MA) on the surface of the colorant particle 10. The particle 10 (including the additive (MA)) is then charged by a charge director. As used herein, the term “charge director” refers to a material that, when used, facilitates charging of the colorant particles. In an example, the charge director is basic and reacts with the acid modified colorant particle 10 to negatively charge the particle 10. In other words, the charging of the particle 10 is accomplished via an acid-base reaction between the charge director and the acid-modified particle 10 surface. It is to be understood that the charge director may also be used in the electronic ink to prevent undesirable aggregation of the colorant in the carrier fluid.

The charge director may be selected from small molecules or polymers that are capable of forming reverse micelles in the non-polar carrier fluid. Such charge directors are generally colorless and tend to be dispersible or soluble in the carrier fluid.

In a non-limiting example, the charge director is selected from a neutral and non-dissociable monomer or polymer such as, e.g., a polyisobutylene succinimide amine, which has a molecular structure as follows:

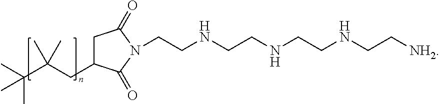

where n is selected from a whole number ranging from 15 to 100.

Another example of the charge director includes an ionizable molecule that is capable of disassociating to form charges. Non-limiting examples of such charge directors include sodium di-2-ethylhexylsulfosuccinate and dioctyl sulfosuccinate. The molecular structure of dioctyl sulfosuccinate is as follows:

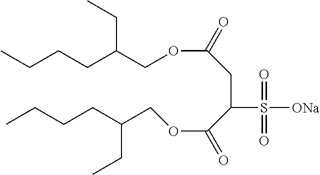

Yet another example of the charge director includes a zwitterion charge director such as, e.g., Lecithin. The molecular structure of Lecithin is as, shown as follows:

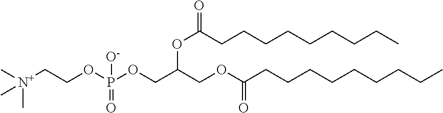

Still another example of the charge director includes a non-chargeable, neutral molecule that cannot disassociate or react with an acid or a base to form charges. This charge director may advantageously be used in embodiments where the colorant particle is charged via adsorption of reverse micelles on the surface of the colorant particle. Such will be described in further detail below. A non-limiting example of such a charge director includes fluorosurfactants having the following molecular structure:

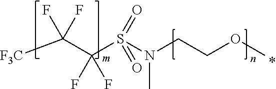

where m is selected from a whole number ranging from 10 to 150, n is selected from a whole number ranging from 5 to 100, and * refers to the repeating base unit.

The charge directors provided above are a few examples of suitable materials that may be used in the various embodiments of the instant disclosure. It is to be understood that other materials may also be used as charge directors, an example of which includes a dispersant. Some non-limiting examples of dispersants that may be used as a charge director include hyperdispersants of the SOLSPERSE® family manufactured by Lubrizol Corp, Wickliffe, Ohio, (e.g., SOLSPERSE® 3000, SOLSPERSE® 8000, SOLSPERSE® 9000, SOLSPERSE® 11200, SOLSPERSE® 13840, SOLSPERSE® 16000, SOLSPERSE® 17000, SOLSPERSE® 18000, SOLSPERSE® 19000); dispersants manufactured by Chevron Oronite Co., San Ramon, Calif., such as OLOA 11000, OLOA 11001, and OLOA 11002; and lubricant dispersants manufactured by Lubrizol Corp., Wickliffe, Ohio, such as LZ2155, OS13709, OS14179, OS13309, and OS45479.

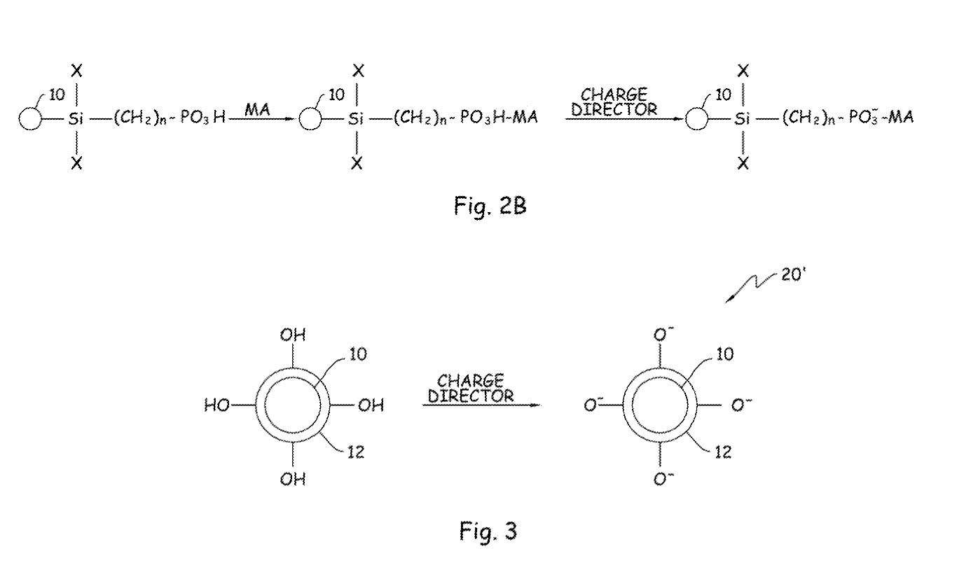

Some specific examples of mechanisms for forming the negatively charged colorant particles 20 for the electronic ink are generally shown in FIGS. 2A and 2B. Such examples follow the same basic mechanism depicted in FIG. 1.

Referring now to the example shown in FIG. 2A, the surface of the pigment particle 10 is acid modified with PO₃H using a substituted benzene derivative as the spacing group (SG) that has the following molecular structure:

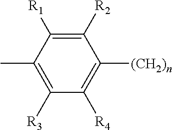

where R₁, R₂, R₃, and R₄ are each independently selected from i) hydrogen, ii) one of a substituted alkyl group, an alkenyl group, an aryl group, an alkyl group, or iii) one of a halogen, —NO₂, —O—R_{d}, —CO—R_{d}, —CO—O—R_{d}, —O—CO—R_{d}, —CO—NR_{d}Rₑ, —NR_{d}Rₑ, —NR_{d}—CO—Rₑ, —NR_{d}—CO—O—Rₑ, NR_{d}—CO—NRₑR_{f}, —SR_{d}, —SO₂—R_{d}, —SO₂—O—R_{d}, —SO₂NR_{d}Rₑ, or a perfluoroalkyl group. The letters R_{d}, Rₑ, and R_{f} are each independently selected from i) hydrogen, or ii) one of a substituted alkyl group, an alkenyl group, an aryl group, or an aralkyl group. Also, the letter n in the benzene derivative may be any whole number ranging from 0 to 6.

Any of the molecular additives (MA) disclosed herein is thereafter connected to the modified pigment particle 10, and then the acid-base reaction between the PO₃H and the charge director takes place to form the negatively charged pigment particle 20.

In the example shown in FIG. 2B, the surface of the pigment particle 10 is modified with PO₃H as the acid functional group (AFG) using an aliphatic chain derivative as the spacing group (SG), which has the following molecular structure:

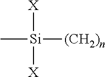

where X represents a halogen, a methoxy group, an ethoxy group, or another alkyloxy group, and the letter n represents any whole number ranging from 0 to 6. This particular acid functional group (AFG) may be suitable for modifying the surface of silica coated pigments (which embodiment will be disclosed in further detail below).

Again, any of the molecular additives (MA) disclosed herein is thereafter connected to the modified pigment particle 10, and then the acid-base reaction between the PO₃H and the charge director takes place to form the negatively charged pigment particle 20.

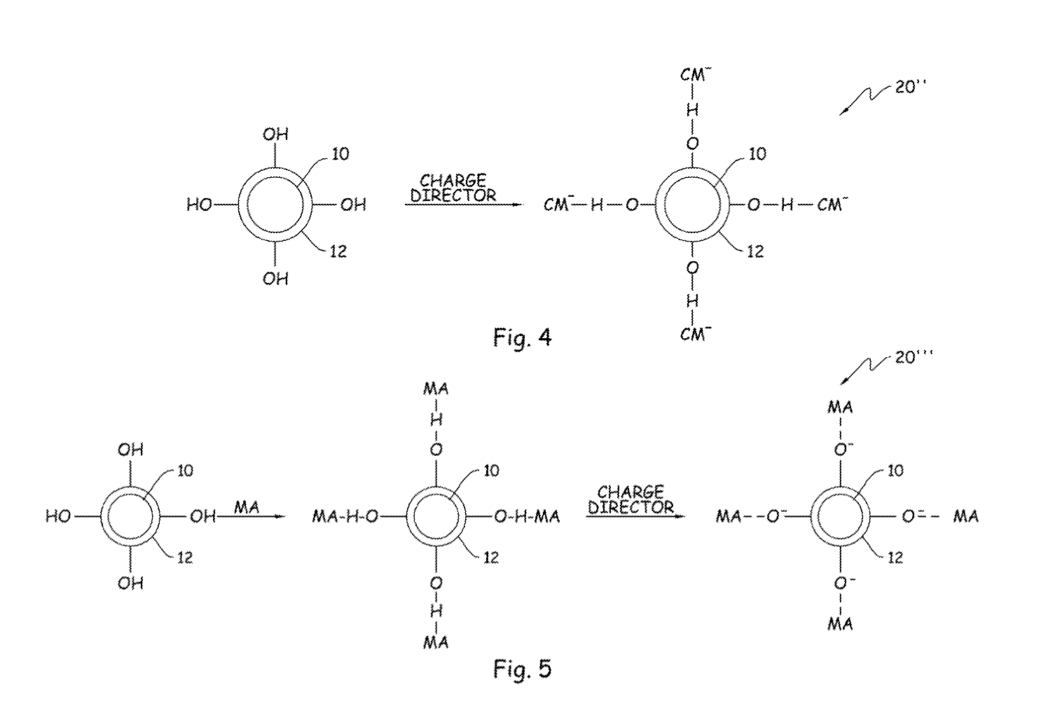

Another embodiment of the electronic ink also includes a negatively charged, non-conductive black colorant dispersion in a non-polar carrier fluid. In this embodiment of the electronic ink, the colorant is selected from carbon black pigment particles coated with a thin (e.g., from about 3 nm to about 100 nm) layer of silica (SiO₂). In an example, the silica is continuously/substantially continuously coated around the particle surface. In another example, the particle surface is partially covered by the silica coating (such as, e.g., in patches). Such particles may be obtained using various fabrication methods such as, e.g., those disclosed in Yuan, J., et al., Journal of Sol-Gel Science and Technology, 2005, 36, 265-274; Bignon, P., et al., U.S. Pat. No. 4,808,239; and Xenopoulos, C., et al. (U.S. Patent Publication No. 2008/0261024). The silica generally acts as an insulating layer on the particle 10 surface and provides —OH functional groups that may be directly charged by the charge director via i) an acid/base reaction mechanism (as shown by the mechanism depicted in FIG. 3), or ii) adsorption of charged micelles or co-micelles (as shown by the mechanism depicted in FIG. 4). In some embodiments, adsorption of a molecular additive (MA, such as those described herein) to the silica coated particle 10 may be accomplished in order to render the particle 10 more chargeable via the acid/base reaction (as shown by the mechanism depicted in FIG. 5). These mechanisms will be described in further detail below.

The non-polar carrier fluid for the instant embodiment of the electronic ink may include one or more of the solvents listed above.

In a non-limiting example, the electronic ink further includes one or more dispersion agents configured to disperse the carbon black pigment particles in the non-polar carrier fluid. Such dispersion agents may also be used to improve the performance of the ink such as, e.g., ink stability, color density, switching speed, and/or the like. The dispersion agent may also be used in the ink as a charge control agent, which is believed to help maintain the negative charge of the colorant once the colorant is charged. The dispersion agent may be selected from any polymeric surfactants that are capable of interacting with the colorant particle to improve the zeta potential of the electronic ink. Some non-limiting examples of suitable dispersion agents include various hyper-dispersants manufactured by Lubrizol Corp., Wickliffe, Ohio, such as, e.g., SOLSPERSE® 3000, SOLSPERSE® 5000, SOLSPERSE® 8000, SOLSPERSE® 11000, SOLSPERSE® 12000, SOLSPERSE® 17000, SOLSPERSE® 19000, SOLSPERSE® 21000, SOLSPERSE® 20000, SOLSPERSE® 27000, and SOLSPERSE® 43000 and/or various dispersants manufactured by Petrolite Corp., St. Louis, Mo., such as, e.g., CERAMAR™ 1608 and CERAMAR™ X-6146.

An example of a mechanism for forming the negatively charged silica coated carbon black pigment 20′ via an acid-base reaction is depicted in FIG. 3. The mechanism includes reacting the hydroxyl (—OH) functional groups present on the surface of the silica coating (identified by reference numeral 12) with a charge director to form negatively charged carbon black pigment particles 20′. Such particles 20′ are thereafter stabilized using one or more of the dispersants described above (not shown in FIG. 3).

An example of a mechanism for forming another embodiment of the negatively charged silica coated carbon black pigment 20″ by adsorption of negatively charged micelles (CM⁻) is depicted in FIG. 4. This mechanism includes interacting the proton (i.e., the hydrogen) portion of the hydroxyl functional group on the surface of the silica 12 with the negatively charged micelles (CM³¹) formed from the charge director. More specifically, the reverse micelles (CM⁻) are formed from the charge director when the charge director is added to the non-polar carrier fluid. The negatively charged micelles (CM⁻) adsorb on the silica surface 12 and negatively charge the pigment particle 10. Again, the particle 20″ may also be stabilized using one or more dispersants (not shown in FIG. 4).

It is to be understood that the charge director is selected based, at least in part, on whether the negatively charged colorant particles are formed via an acid/base reaction or via adsorption of negatively charged micelles. The selection of the charge director typically depends, at least in part, on the nature of the charge director and the surface chemistry of the colorant particles. Some charge directors may, in some instances, be used for both the acid/base reaction and the formation and adsorption of negatively charged micelles mechanisms.

An example of a mechanism for forming still another embodiment of the negatively charged silica coated carbon black pigment 20′″ is depicted in FIG. 5. This mechanism includes first interacting the silica coated carbon black particle 10, 12 with a molecular additive (MA). Intermolecular hydrogen bonding causes the sterically hindered molecular additive to attach to the surface of the coated particle 10, 12. More particularly, the oxygen or nitrogen atoms of the molecular additive (MA) hydrogen bond to the hydroxyl functional groups on the carbon black surface. This particle 10 (having the silica coating 12 and molecular additive (MA) attached thereto) is thereafter charged using a charge director. It is believed that the adsorption of the molecular additive (MA) sterically hinders the colorant particle, increases the hydrophobicity of the colorant particle, and increases the acidity of the hydroxyl group(s) of the colorant particle, thereby improving the chargeability and the dispersibility of the silica coated particle 10, 12. Due, at least in part, to the increased acidity of the hydroxyl group(s) of the colorant particle, such particles 10, 12 are thus more amenable to the acid/base reaction that takes place with the charge director.

The negatively charged colorant particles may be formed using any of the mechanisms disclosed hereinabove. It is to be understood that, in some instances, two or more of the mechanisms may be combined to form the negatively charged colorant particles. For example, the colorant particles may be formed by adsorption of the molecular additive in combination with adsorption of charged micelles.

Further, it is to be understood that any of the embodiments of the electronic ink may be made using any suitable dispersion methods known by those skilled in the art. Some non-limiting examples of such methods include grinding, milling, attriting, agitation via a paint-shaker, microfluidizing, ultrasonic techniques, and/or the like.

Still further, the amounts of each of the components used to form the inks disclosed herein may vary, depending at least in part, on the desirable amount to be made, the application in which it will be used, etc.

To further illustrate embodiment(s) of the present disclosure, the following examples are given herein. It is to be understood that these examples are provided for illustrative purposes and are not to be construed as limiting the scope of the disclosed embodiment(s).

### EXAMPLES

### Comparative Example 1

About 2 wt % of a polyisobutylene succinimide and about 1 wt % of a polymeric hyperdispersant was introduced into an isoparaffinic fluid to form a solution. The amount of isoparaffinic fluid used for the solution was an amount sufficient to form the 2 wt % of the polyisobutylene succinimide and 1 wt % of the polymeric hyperdispersant concentrations (i.e., any volume is suitable as long as the final weight percents of the various components are achieved). About 3 wt % of a carboxylic acid functionalized carbon black pigment CB was added to the solution. The mixture yielded an electronic ink that included negatively charged carbon black pigment particles having a size of about 200 nm and a zeta potential of about −20 mV.

The carboxylic acid functionalized carbon black pigment CB used in this example may be made by adding carboxylic acidic propylbenzene diazonium salt (20 mmol) to a suspension of carbon black (10 mmol) in water (50 mL). The resulting mixture is stirred at room temperature for 24 hours. Then the mixture is filtered off and dried in vacuum to afford the acid modified carbon black.

### Example 2

About 2 wt % of a polyisobutylene succinimide and about 1 wt % of a polymeric hyperdispersant was introduced into an isoparaffinic fluid to form a solution (similar to Example 1). About 3 wt % of an acid functionalized carbon black pigment CB was added to the solution. About 2 wt % of pentaerythritol tetraacrylate was added to the mixture. The product yielded an electronic ink having negatively charged carbon black pigment particles having a size of about 170 nm and a zeta potential of about −40 mV (which is higher than that of the electronic ink made by the method disclosed in Comparative Example 1).

### Example 3

About 2 wt % of a polyisobutylene succinimide and about 1 wt % of a polymeric hyperdispersant was introduced into an isoparaffinic fluid to form a solution (similar to Example 1). About 3 wt % of an acid functionalized carbon black pigment CB was added to the solution. About 2 wt % of ethoxylated(3) trimethylolpropane triacrylate was added to the mixture. The product yielded an electronic ink having negatively charged carbon black pigment particles having a size of about 150 nm and a zeta potential of about −40 mV (which is also higher than that of the electronic ink made by the method disclosed in Comparative Example 1).

### Example 4

About 2 wt % of a polyisobutylene succinimide and about 1 wt % of a polymeric hyperdispersant was introduced into an isoparaffinic fluid to form a solution (similar to Example 1). About 3 wt % of an acid functionalized carbon black pigment CB was added to the solution. About 2 wt % of trimethylolpropane ethyoxylate (1EO/OH) methyl ether diacrylate was added to the mixture. The product yielded an electronic ink having negatively charged carbon black pigment particles having a size of about 160 nm and a zeta potential of about −30 mV (which is again higher than that of the electronic ink made by the method disclosed in Comparative Example 1).

### Comparative Example 5

About 1 wt % of a polyisobutylene succinimide and about 10 wt % of a polymeric hyperdispersant was introduced into an isoparaffinic fluid to form a solution. About 10 wt % of silica coated carbon black pigment CB was added to the solution and the resultant mixture was bead milled with ZrO₂ milling beads. The milled mixture was diluted to about 5 wt % of silica coated carbon black CB by slow addition of the isoparaffinic fluid and then was subjected to filtration by a vacuum to yield an electronic ink having negatively charged carbon black pigment particles having a size of about 220 nm and a zeta potential of about −20 mV.

### Example 6

About 1 wt % of a polyisobutylene succinimide and about 10 wt % of a polymeric hyperdispersant was introduced into an isoparaffinic fluid to form a solution. About 10 wt % of silica coated carbon black pigment CB was added to the solution and the resultant mixture was bead milled with ZrO₂ milling beads. The milled mixture was diluted to about 5 wt % of silica coated carbon black CB by slow addition of an isoparaffinic fluid and then was subjected to filtration by a vacuum. About 2 wt % of ethoxylated(3) trimethylolpropane triacrylate was added to the diluted mixture. The product yielded an electronic ink having negatively charged carbon black pigment particles having a size of about 190 nm and a zeta potential of about −30 mV (which is higher than that of the electronic ink formed by the method disclosed in Comparative Example 5).

Examples 2-4 and 6 each included one of the molecular additives disclosed herein, while the Comparative Examples 1 and 5 did not include such molecules. As illustrated in the Examples, the zeta potential of those inks including the molecular additives disclosed herein was higher than the zeta potential of the Comparative Examples.

While several embodiments have been described in detail, it will be apparent to those skilled in the art that the disclosed embodiments may be modified. Therefore, the foregoing description is to be considered exemplary rather than limiting.

What is claimed is:

1. An electronic ink, comprising:
a non-polar carrier fluid;
a plurality of negatively charged colorant particles dispersed in the non-polar carrier fluid, each of the negatively charged colorant particles including a surface modified with an acidic functional group;
a small molecular additive having at least two branches, each of which includes nitrogen or oxygen atoms; and
a charge director.

2. The electronic ink as defined in claim 1 wherein the plurality of negatively charged colorant particles has an average particle size ranging from about 1 nm to about 10 μm.

3. The electronic ink as defined in claim 1 wherein the surface of each of the colorant particles further includes a spacing group configured to connect the colorant particle with the acidic functional group, the spacing group being selected from benzenes, substituted benzenes, naphthalenes, molecules including aliphatic chains, substituted naphthalenes, hetero-aromatic structures, inorganic coatings, and combinations thereof.

4. The electronic ink as defined in claim 1 wherein the acidic functional group is selected from OH, SH, COOH, CSSH, COSH, SO₃H, PO₃H, OSO₃H, OPO₃H, or combinations thereof.

5. The electronic ink as defined in claim 1 wherein the small molecular additive is selected from: 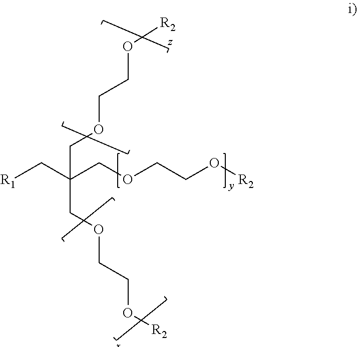
wherein:
R₁ and R₂ are each independently selected from an alkyl group, a branched alkyl group, an aliphatic group, an aromatic acyl group, an alkenyl group, and a branched alkenyl group; and
x, y, and z are each selected from any whole number ranging from 0 to 10; 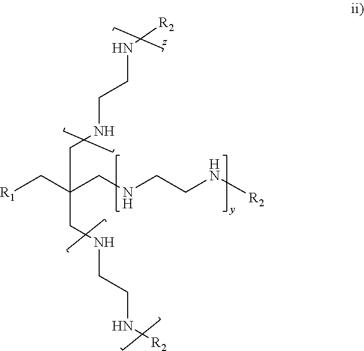
wherein:
R₁ and R₂ are each independently selected from an alkyl group, a branched alkyl group, an aliphatic group, an aromatic acyl group, an alkenyl group, and a branched alkenyl group; and
x, y, and z are each selected from any whole number ranging from 0 to 10; 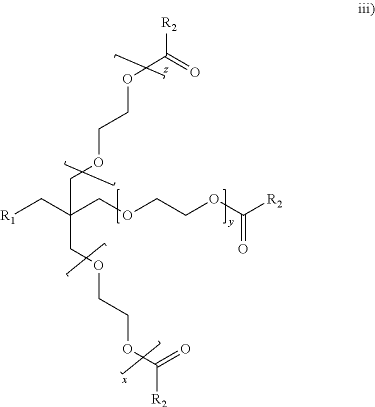
wherein:
R₁ and R₂ are each independently selected from an alkyl group, a branched alkyl group, an alkenyl group, or a branched alkenyl group; and
x, y, and z are each selected from a whole number ranging from 0 to 10; 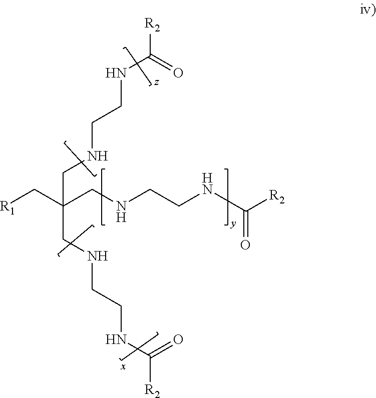
wherein:
R₁ and R₂ are each independently selected from an alkyl group, a branched alkyl group, an alkenyl group, or a branched alkenyl group; and
x, y, and z are each selected from a whole number ranging from 0 to 10; 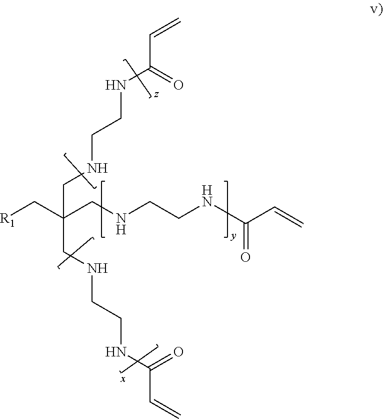
wherein:
R₁ is selected from an alkyl group, a branched alkyl group, an alkenyl group, or a branched alkenyl group; and
x, y, and z are selected from a whole number ranging from 0 to 10; and 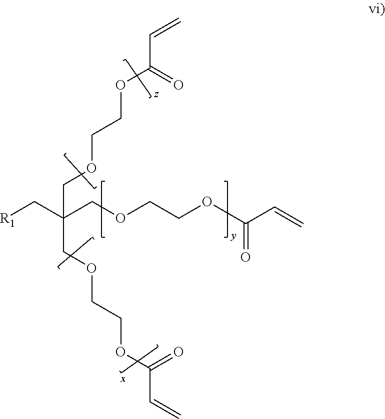
wherein:
R₁ is selected from an alkyl group, a branched alkyl group, an alkenyl group, or a branched alkenyl group; and
x, y, and z are selected from a whole number ranging from 0 to 10.

6. The electronic ink as defined in claim 1 wherein the colorant particles are selected from yellow pigment particles, green pigment particles, brown pigment particles, cyan pigment particles, blue pigment particles, magenta pigment particles, red pigment particles, orange pigment particles, white pigment particles, spot-color pigment particles, and black pigment particles.

7. The electronic ink as defined in any claim 1 wherein a zeta potential of the ink ranges from greater than −20 to about −200 mV.

8. The electronic ink as defined in claim 1 wherein the non-polar carrier fluid is a non-polar solvent is selected from perchloroethylene, halocarbons, cyclohexane, dodecane, mineral oil, isoparaffinic fluids, siloxanes, and combinations thereof.

9. The electronic ink as defined in claim 1 wherein the small molecular additive has a molecular weight that is less than 2000.

10. An electronic ink, comprising:
a non-polar carrier fluid;
a charge director capable of forming reverse miscelles in the non-polar solvent;
a plurality of negatively charged, non-conductive carbon black colorant particles dispersed in the non-polar solvent, each of the negatively charged, non-conductive carbon black colorant particles including:
a core colorant particle having a surface; and
an inorganic insulating coating established on the surface of the core colorant particle, the inorganic insulating coating including at least one functional group that is chargeable by i) an acid-base reaction with the charge director, or ii) adsorption of at least one of the reversed micelles formed from the charge director on the surface; and
a small molecular additive having at least two branches, each of which includes nitrogen or oxygen atoms.

11. The electronic ink as defined in claim 10 wherein the charge director is selected from i) a neutral and non-dissociable monomer, ii) a neutral and non-dissociable polymer, iii) an ionizable molecule that is capable of disassociating to form charges, iv) a zwitterion, and v) a non-chargeable, neutral molecule that cannot disassociate or react with an acid or a base to form charges.

12. The electronic ink as defined in claim 10 wherein the small molecular additive renders the plurality of colorant particles more chargeable by the acid-base reaction with the charge director.

13. The electronic ink as defined in claim 12 wherein the small molecular additive is selected from: 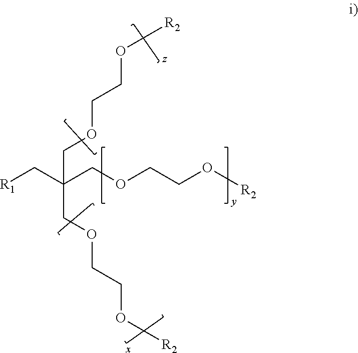
wherein:
R₁ and R₂ are each independently selected from an alkyl group, a branched alkyl group, an aliphatic group, an aromatic acyl group, an alkenyl group, and a branched alkenyl group; and
x, y, and z are each independently selected from any whole number ranging from 0 to 10; 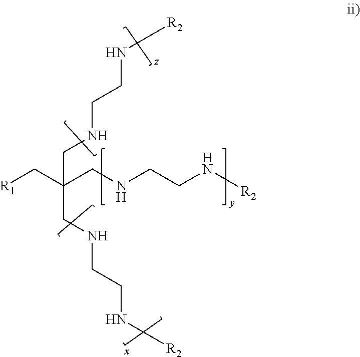
wherein:
R₁ and R₂ are each independently selected from an alkyl group, a branched alkyl group, an aliphatic group, an aromatic acyl group, an alkenyl group, and a branched alkenyl group; and
x, y, and z are each independently selected from any whole number ranging from 0 to 10; 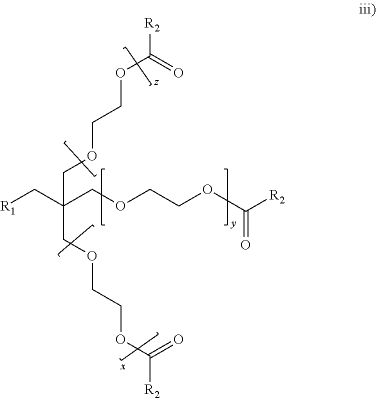
wherein:
R₁ and R₂ are each independently selected from an alkyl group, a branched alkyl group, an alkenyl group, and a branched alkenyl group; and
x, y, and z are each independently selected from a whole number ranging from 0 to 10; 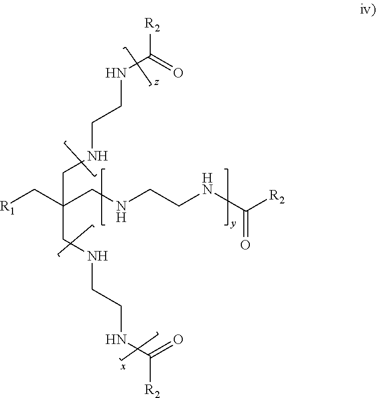
wherein:
R₁ and R₂ are each selected from an alkyl group, a branched alkyl group, an alkenyl group, and a branched alkenyl group; and
x, y, and z are each selected from a whole number ranging from 0 to 10; 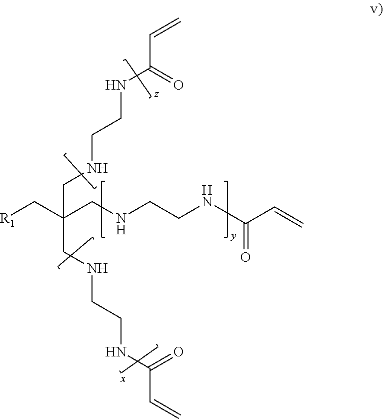
wherein:
R₁ is selected from an alkyl group, a branched alkyl group, an alkenyl group, and a branched alkenyl group; and
x, y, and z are each independently selected from a whole number ranging from 0 to 10; and 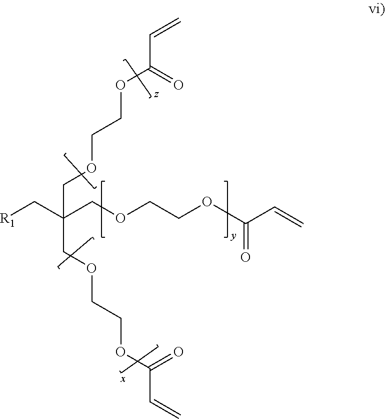
wherein:
R₁ is selected from an alkyl group, a branched alkyl group, an alkenyl group, and a branched alkenyl group; and
x, y, and z are each independently selected from a whole number ranging from 0 to 10.

14. The electronic ink as defined in claim 10 wherein the small molecular additive has a molecular weight that is less than 2000.

15. The electronic ink as defined in claim 10, further comprising a dispersing agent configured to interact with the colorant particle to improve a zeta potential of the electronic ink.

16. The electronic ink as defined in claim 10 wherein the electronic ink is configured to be used in an electro-optical display using electrophoresis, electro-convective flow, other electrokinetic effects, or combinations thereof.

17. A small molecular additive for an electronic ink selected from one of the following formulas: 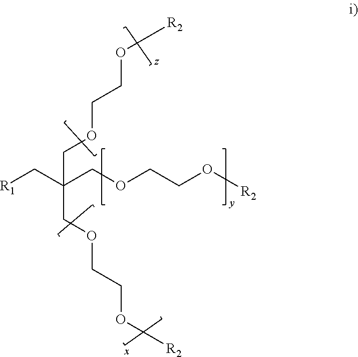
wherein:
R₁ and R₂ are each independently selected from an alkyl group, a branched alkyl group, an aliphatic group, an aromatic acyl group, an alkenyl group, and a branched alkenyl group; and
x, y, and z are each independently selected from any whole number ranging from 0 to 10; 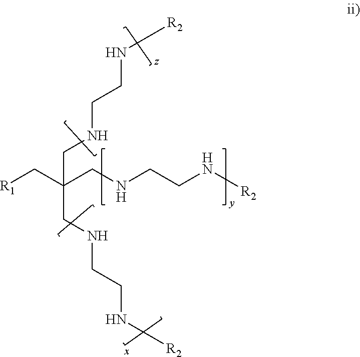
wherein:
R₁ and R₂ are each independently selected from an alkyl group, a branched alkyl group, an aliphatic group, an aromatic acyl group, an alkenyl group, and a branched alkenyl group; and
x, y, and z are each independently selected from any whole number ranging from 0 to 10; 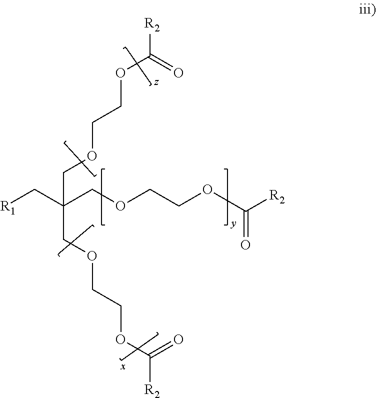
wherein:
R₁ and R₂ are each independently selected from an alkyl group, a branched alkyl group, an alkenyl group, or a branched alkenyl group; and
x, y, and z are each independently selected from a whole number ranging from 0 to 10; 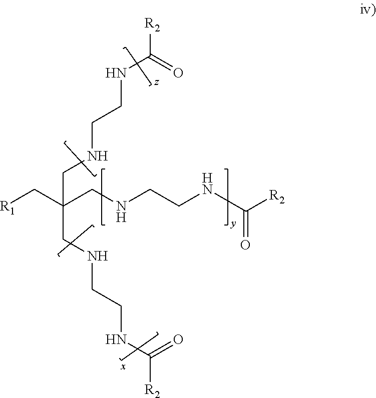
wherein:
R₁ and R₂ are each independently selected from an alkyl group, a branched alkyl group, an alkenyl group, or a branched alkenyl group; and
x, y, and z are each independently selected from a whole number ranging from 0 to 10; 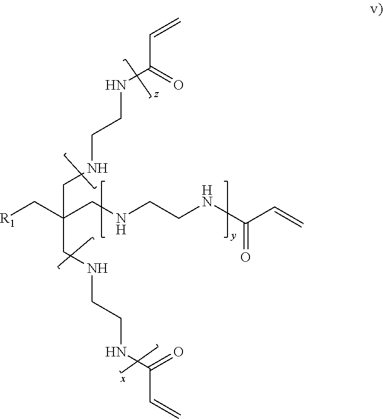
wherein:
R₁ is selected from an alkyl group, a branched alkyl group, an alkenyl group, or a branched alkenyl group; and
x, y, and z are each independently selected from a whole number ranging from 0 to 10; and 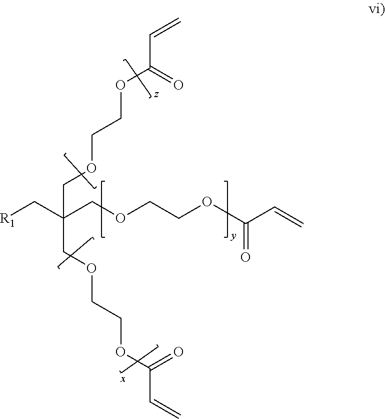
wherein:
R₁ is selected from an alkyl group, a branched alkyl group, an alkenyl group, or a branched alkenyl group; and
x, y, and z are each independently selected from a whole number ranging from 0 to 10.

18. The small molecular additive as defined in claim 17 wherein the molecular additive is configured to be adsorbed on a surface of a pigment included in the electronic ink, and wherein the molecular additive is configured to sterically hinder the pigment.
